(19) United States
(12) Patent Application Publication
(10) Pub. No.: US 2006/0134228 A1
(43) Pub. Date: Jun. 22, 2006

(54) Medicine for prevention of and treatment for amyloidosis

(75) Inventors: Hirofumi Kai, Kumamoto (JP); Yukio Ando, Kumamoto (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Nipro Corporation, Osaka-shi, Osaka (JP)

(21) Appl. No.: 10/514,335

(22) PCT Filed: May 13, 2003
(86) PCT No.: PCT/JP03/05963

§ 371 Date: Jan. 18, 2005

(30) Foreign Application Priority Data
May 14, 2002 (JP) 2002-137944

(51) Int. Cl. A61K 33/24; A61K 31/555

(52) U.S. Cl. 424/655; 514/184

(57) ABSTRACT

A pharmaceutical for the prophylaxis and/or treatment of amyloidosis such as familial amyloid polyneuropathy is provided. A pharmaceutical for the prophylaxis and/or treatment of amyloidosis that contains a compound containing the trivalent chromium ion, such as a fatty acid chromium salt, as an active ingredient, and that is capable of suppressing the decomposition of mutant transthyretin in amyloidosis.

### TECHNICAL FIELD

The present invention relates to a medicine for the prophylaxis and/or treatment of amyloidosis.

### BACKGROUND ART

Amyloidosis refers to a group of diseases wherein a protein having a β-sheet structure polymerizes to form insoluble fibrils called amyloid (amyloid), which deposit in the body and cause tissue injuries. German pathologist Virchow discovered that tissue specimens of amyloidosis stain purple with iodine. From this result, Virchow considered the substance that had deposited on the tissue as a polysaccharide, named this as “starch-like substance”, namely “amyloid”, and proposed to call the pathologic condition caused by amyloid deposition as amyloidosis. By subsequent studies, it has been found that the major component of amyloid is the protein that forms the fiber thereof, and that amyloid contains the serum amyloid P component, glycosaminoglycan and the like. At present, amyloid is defined as “comprising an aggregate of non-branched fibrils of 8-15 nm width that stains orange-red with Congo Red Stain and exhibits green vividly shining double refraction when examined under the polarization microscope”.

The illness caused by amyloid, that is, amyloidosis, includes various diseases such as Alzheimer's disease, Creutzfeldt-Jacob disease (mad cow disease), Huntington's disease or hereditary diseases. In particular, attention-attracting familial amyloid polyneuropathy (FAP) is a form of amyloidosis characterized by systemic amyloid deposition in organs such as peripheral nerves, autonomic nerves, kidneys, skin and mucosa, and showing introgression by autosomal dominant inheritance. Since a case of FAP was reported for the first time in a Portuguese in 1952, similar case reports have been presented from worldwide. The amyloid fiber that causes FAP is composed of a mutant of transthyretin (TTR), which is a serum protein. The normal TTR usually occurs as a tetramer, whereas mutant TTRs have been shown to be low in structural stability and likely to undergo a structural change from the tetramer to the monomer.

A form of FAP of relatively high incidence in our country is type I FAP, which has a mutant TTR of the Val30Met type as the causal protein. It is said that mutant TTR proteins are lower in structural stability than normal TTR proteins, and that the β-sheet structures in the molecule thereof associate with each other to form insoluble amyloid fiber and deposit in tissue. Major symptoms of type I FAP are multiple neurosis accompanied by sensory disorders that ascend laterally symmetrically from the lower extremity ends, and autonomic disorders (alternating diarrhea and constipation, orthostatic hypotension, dysuria etc.); these are all caused by nerve injuries due to amyloid. Subsequently, amyloid deposition in the heart, kidneys and digestive tract becomes considerable and causes dysfunction of these organs. With no radical therapy to remove once deposited amyloid fiber, FAP is a disease of poor prognosis that develops in the last half of the 20s to the 30s, leads to walking disability in 10 years, and is fatal due to infectious disease, heart failure, renal insufficiency and the like over a course of 10-20 years, and is an intractable disease designated as a specified disease by the Ministry of Health and Welfare.

Currently, although treatment of FAP is conducted mainly by symptomatic therapies, liver transplantation is also adopted as being presumably the most etiotropic therapy. This is a therapeutic method based on the expectation that progression of the condition could be stopped by replacing the patient's liver with a donor liver that produces the normal TTR, because the primary TTR-producing organ is the liver. However, this therapy is not a generally adoptable therapeutic method because an additional burden is exerted on the donor.

The object of the present invention resides in providing a medicine for the prophylaxis and/or treatment of amyloidosis. Particularly, the object of the present invention is to provide a medicine for the prophylaxis and/or treatment of familial amyloid polyneuropathy (FAP), and to provide a medicine to prevent the onset of sensory disorders and autonomic disorders caused by amyloidosis, and a medicine for treatment that enables prevention of progression of the above-described sensory disorders and autonomic disorders, etc.

### DISCLOSURE OF THE INVENTION

The present inventors conducted extensive investigations to accomplish the above-described object, and found that compounds containing the trivalent chromium ion, such as fatty acid chromium salts, have an excellent effect as a medicine for the prophylaxis and/or treatment of amyloidosis, and completed the present invention.

Accordingly, the present invention provides a medicine for the prophylaxis and/or treatment of amyloidosis, which comprises a compound containing a trivalent chromium ion as an active ingredient. In a preferred embodiment of the invention, the above-described pharmaceutical wherein amyloidosis is familial amyloid polyneuropathy (FAP), the above-described pharmaceutical wherein the compound containing the trivalent chromium ion is a fatty acid chromium salt, and the above-described pharmaceutical wherein the fatty acid is picolinic acid, are provided.

From another viewpoint, the present invention provides a medicine to suppress decomposition of mutant transthyretin in amyloidosis, which comprises a compound containing a trivalent chromium ion as an active ingredient, and a medicine to stabilize mutant transthyretin in amyloidosis, which comprises a compound containing a trivalent chromium ion as an active ingredient. In a preferred embodiment of this invention, the above-described pharmaceutical which is an inhibitor of the transthyretin precursor decomposition reaction promotion by the aluminum ion, the above-described pharmaceutical wherein amyloidosis is familial amyloid polyneuropathy (FAP), the above-described pharmaceutical wherein the compound containing the trivalent chromium ion is a fatty acid chromium salt, and the above-described pharmaceutical wherein the fatty acid is picolinic acid, are provided.

Also, a mutant transthyretin decomposition suppressor comprising a compound containing the trivalent chromium ion as an active ingredient, and a mutant transthyretin stabilizer comprising a compound containing the trivalent chromium ion as an active ingredient, are provided by the present invention.

From still another viewpoint, use of a compound containing the trivalent chromium ion for production of the above-described pharmaceutical, a prophylactic and/or therapeutic method for amyloidosis that includes a step wherein a prophylactically and/or therapeutically effective amount of a compound that contains the trivalent chromium ion is administered to a mammalian animal, including human, and a method to suppress decomposition of mutant transthyretin in amyloidosis that includes a step wherein a prophylactically and/or therapeutically effective amount of a compound that contains the trivalent chromium ion is administered to a mammalian animal, including a human, are provided by the present invention.

As an active ingredient for the pharmaceutical of the present invention, one or two kinds or more of a compound that contains the trivalent chromium ion can be used. Although the kind of the compound that contains the trivalent chromium ion is not limited, the compound is preferably a fatty acid salt, for example, so that it is readily absorbable from the gastrointestinal tract. As examples of the salt-forming fatty acid, butyric acid, picolinic acid, pyruvic acid etc., which are readily metabolizable via in vivo metabolic pathways, are preferred, which, however, are not to be construed as limiting. Of these, picolinic acid, which is non-toxic, and whose rate of gastrointestinal absorption is rapid, is most preferable. Also, as the pharmaceutical of the present invention, an optionally chosen mixture containing a compound that contains the trivalent chromium ion may also be used. Examples of such a mixture include mixtures like natural product extracts and beer fermentation cake.

The pharmaceutical of the present invention can be used as a medicine for the prophylaxis and/or treatment of amyloidosis. As examples of the amyloidosis to which the pharmaceutical of the present invention is applied, neurodegenerative diseases, such as Alzheimer's disease, bovine spongiform encephalopathy (BSE), Creutzfeldt-Jacob disease (CJD), laughing death syndrome, scrapie, variant Creutzfeldt-Jacob disease in humans and the like, as well as familial amyloid polyneuropathy (FAP), can be mentioned. Of these, familial amyloid polyneuropathy is a particularly suitable target of the pharmaceutical of the present invention.

Although there is no reason for adhering to any particular theory, the action mechanism of the pharmaceutical of the present invention can be explained as follows. Transthyretin (TTR), which is a serum protein, occurs as a tetrameric protein in the serum, and this tetramer is decomposed to monomers by the action of an unidentified catalyst or enzyme and the like. The majority of these monomers are decomposed, some of which go into in vivo metabolic pathways and is excreted as urine to outside of the body; some of the monomers undergo rearrangement to form a sheet structure, and this β-sheet structure polymerizes linearly to produce insolubilized fibrous amyloid fiber. Mutant transthyretin is prone to be decomposed from the tetrameric protein to the monomer; as a result, amyloid fiber production increases.

The present inventors conducted research into the mechanism by which amyloid fiber is produced from transthyretin, and confirmed the presence of individuals that do not develop FAP despite that they have mutant transthyretin. Hence, on the basis of the idea that environmental factors are involved in the production of the above-described amyloid fiber, the inventors ascertained that metal ions, out of environmental factors, are profoundly involved in amyloid fiber production. For example, as shown in the Examples, the aluminum ion possesses an action to promote the reaction of decomposing the transthyretin tetramer to the monomer. On the other hand, the trivalent chromium ion possesses an action to inhibit the reaction of decomposing the transthyretin tetramer to produce the monomer; as a result, amyloidosis can be prevented and/or treated by suppressing the reaction of producing amyloid fiber from mutant transthyretin.

For example, for mildly symptomatic patients out of patients who have already developed FAP, progression and exacerbation of symptoms can be prevented by administering the pharmaceutical of the present invention. There are some cases wherein a therapeutic effect can be expected even in seriously symptomatic patients. Also, individuals having mutant transthyretin are potential patients who are very likely to develop FAP with aging; onset of FAP can be prevented by administering the pharmaceutical of the present invention.

As the pharmaceutical of the present invention, a compound that contains the trivalent chromium ion, which is an active ingredient, may be used as is; however, usually, it is desirable that a dosage form of a pharmaceutical composition containing both a compound that contains the trivalent chromium ion or a mixture containing it, which is an active ingredient, and one or two kinds or more of additives for pharmaceutical making, be prepared and used.

In preventing and treating FAP, it is also desirable that the pharmaceutical of the present invention be used in combination not only with the trivalent chromium ion, but also with a non-steroidal anti-inflammatory drug that stabilizes the transthyretin tetramer, for example, diflunisal.

As examples of a composition for pharmaceutical use suitable for oral administration, tablets, capsules, powders, fine granules, granules, liquids, and syrups and the like can be mentioned; as examples of a pharmaceutical composition suitable for parenteral administration, injections, drip infusions, suppositories, inhalants, eye drops, nasal drops, ointments, creams, plasters, transdermally absorbable agents, or transmucosally absorbable agents and the like can be mentioned. As examples of additives for preparation making used for production of the above-described pharmaceutical composition, fillers such as lactose and oligosaccharides, disintegrants or disintegration aids, binders, lubricants, coating agents, dyes, diluents, substrates, solvents or solubilizers, isotonizing agents, pH regulators, stabilizers, propellants, and adhesives and the like can be mentioned; these can be appropriately selected according to the form of the pharmaceutical composition by those skilled in the art, and can also be used in combination of two kinds or more.

As a preferred form of the pharmaceutical of the present invention, tablets can be mentioned. Tablet diameter is 2-10 mm or so and thickness is 1-5 mm or so; an active ingredient can be contained at 0.01% by weight or more, preferably 0.1-10% by weigh, per 100% by weight of the tablets. Note that as a medicine that contains chromium picolinate, for example, “Yeast Free Chromium Picolinate” is commercially available as a nutritional supplement in the form of tablets from Vitamin World (NY, USA) Company; this tablet can also be used as is as the pharmaceutical of the present invention.

The dosage and administration frequency and the like for the pharmaceutical of the present invention are not subject to limitation, and can be appropriately selected according to conditions such as patient age, body weight and sex, and the kind and seriousness of the disease, the purpose of prophylaxis or treatment, and the like. Usually, in the case of oral administration, the dosage is 0.01 g to 1 g or so per day for an adult as the amount of active ingredient; the above-described dosage may be administered in several divided portions in a day.

### BRIEF DESCRIPTION OF THE DRAWINGS

FIG. 1 is a drawing showing that the pharmaceutical of the present invention possesses an action to suppress the decomposition from the transthyretin tetramer to the transthyretin monomer, and that on the other hand, aluminum possesses an action to promote the decomposition.

FIG. 2 is a drawing showing that the pharmaceutical of the present invention possesses an action to suppress the decomposition from the normal transthyretin tetramer to the transthyretin monomer, and to suppress amyloid fiber production.

FIG. 3 is a drawing showing that the pharmaceutical of the present invention possesses an action to suppress the decomposition from a mutant transthyretin tetramer to the transthyretin monomer, and to suppress amyloid fiber production.

FIG. 4 is a drawing showing that the pharmaceutical of the present invention possesses an action to increase the thermal stability during structural conversion from the transthyretin tetramer to the transthyretin monomer.

FIG. 5 is a drawing showing that the pharmaceutical of the present invention acts to increase the thermal stability during structural conversion from the transthyretin tetramer to the transthyretin monomer, and to stabilize the transthyretin tetramer structure, even under acidic conditions that destabilize the transthyretin tetramer structure.

FIG. 6 is a drawing showing that the pharmaceutical of the present invention possesses an action to increase the stability of a mutant transthyretin tetramer structure in vivo.

FIG. 7 is a drawing showing that the pharmaceutical of the present invention possesses an action to suppress amyloid fiber production by transthyretin, in cooperation with thyroid hormone, which stabilizes the transthyretin tetramer.

FIG. 8 is a drawing showing that the pharmaceutical of the present invention possesses an action to suppress amyloid fiber production by transthyretin, in cooperation with thyroid hormone, which stabilizes a recombinant transthyretin tetramer.

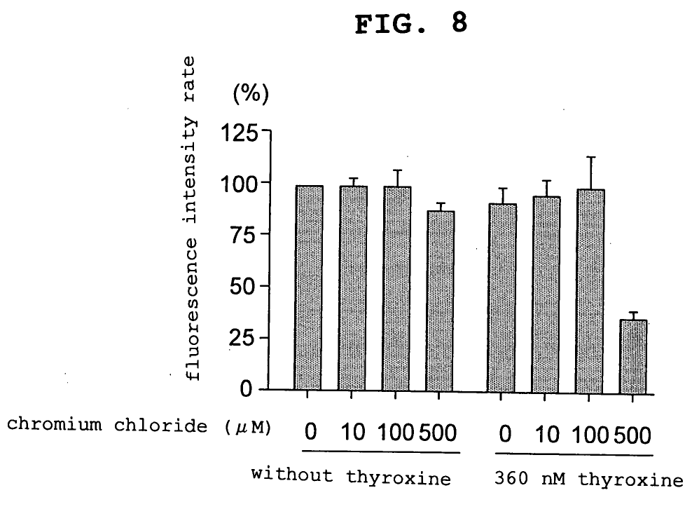

FIG. 9 is a drawing showing that the pharmaceutical of the present invention possesses an action to suppress amyloid fiber production by transthyretin, in cooperation with the anti-inflammatory drug diflunisal, which stabilizes the transthyretin tetramer.

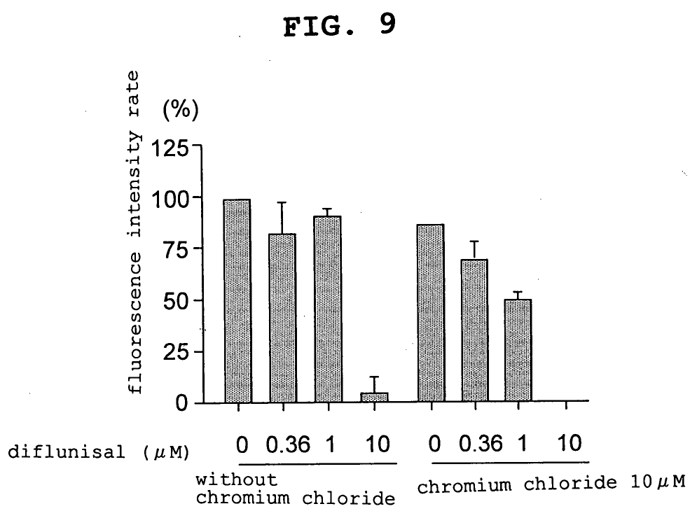

### BEST MODE FOR EMBODYING THE INVENTION

The present invention is hereinafter described more specifically by means of examples, but the scope of the present invention is never limited to the following examples.

### EXAMPLE 1

(1) Amyloid Fiber Production Test

Using transthyretin purified from human serum as a transthyretin-containing solution, the purity thereof was confirmed by electrophoresis. The transthyretin-containing solution (10 mg/mL) was dissolved in a buffer solution to obtain a concentration of 0.2 mg/mL, and chromium chloride (80 μg/mL) was added. The pH of the buffer solution was adjusted to a pH necessary for each experiment using sodium hydroxide or hydrochloric acid. The above-described solution was incubated at 37° C. to produce amyloid fiber.

(2) Measurement of Amount of Amyloid Fiber Produced

Using Thioflavine T (Wako Pure Chemical Industries, Ltd.), which is a fluorescence probe that selectively binds to polymerized β-sheets, the amount of amyloid fiber produced was measured. As a measurement sample, the transthyretin-containing solution after incubation was diluted to obtain a transthyretin concentration of 2.5 μg/mL, supplemented with 10 μM Thioflavine T and 50 mM glycine, adjusted to pH 9.0, and used. Within 1 minute after preparation of the measurement sample, fluorescence at 482 nm excited by 450 nm excitation light was measured with the fluorescence spectrophotometer F-4500 at 25° C. using 1 ml of the sample. Note that both for the excitation side and for the fluorescence side, the measurement was conducted with a 5 nm slit width.

(3) Measurement of Quantitative Ratio of Transthyretin Tetramer and Transthyretin Monomer

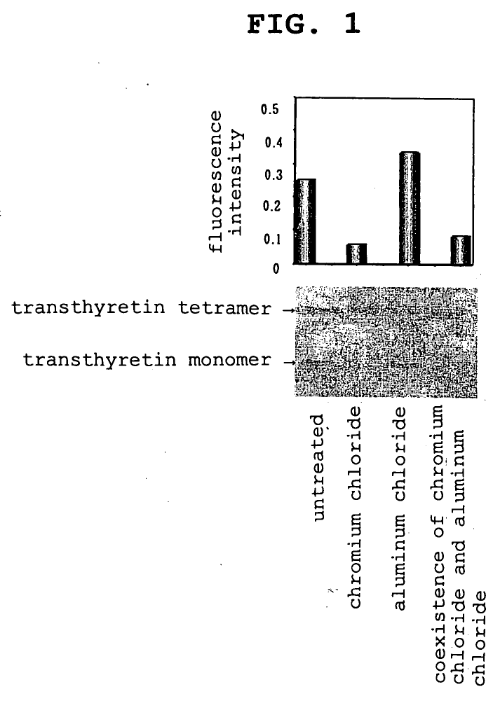

The transthyretin tetramer and the transthyretin monomer were separated by gel separation chromatography, immersed in the Coomassie Brilliant Blue stain, and examined for color developing intensity. Likewise, measurements were conducted for a comparative example wherein the metal compound added to the solution for incubation was changed from chromium chloride to aluminum chloride, and for a control not formulated with chromium chloride. Also, another test was conducted in the same manner, except that the metal compound added to the solution for incubation was replaced with 80 μg/mL chromium chloride and 80 μg/mL aluminum chloride (chromium chloride and aluminum chloride coexisted). Measurement results of fluorescence intensity and the quantitative ratio of the transthyretin tetramer and the transthyretin monomer are shown in FIG. 1.

### EXAMPLE 2

(1) Measurement of Changes in Thermal Stability of Transthyretin in the Presence of the Trivalent Chromium Ion

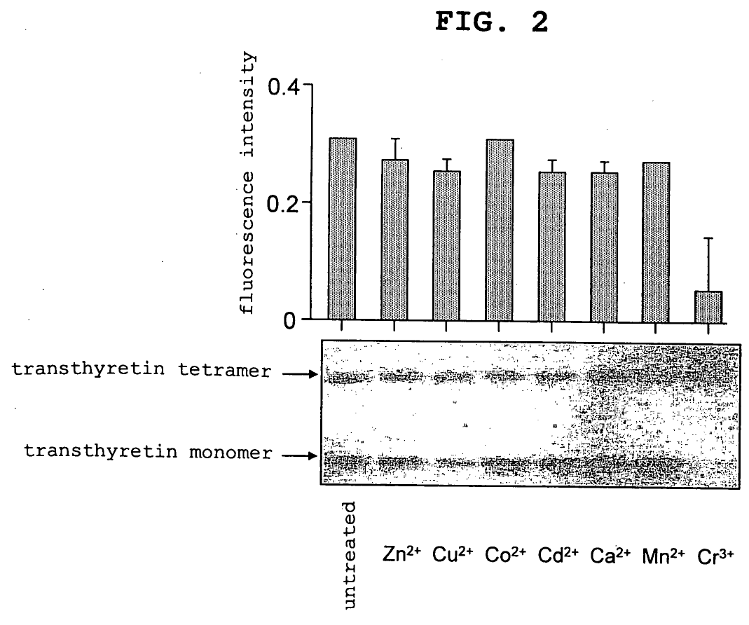

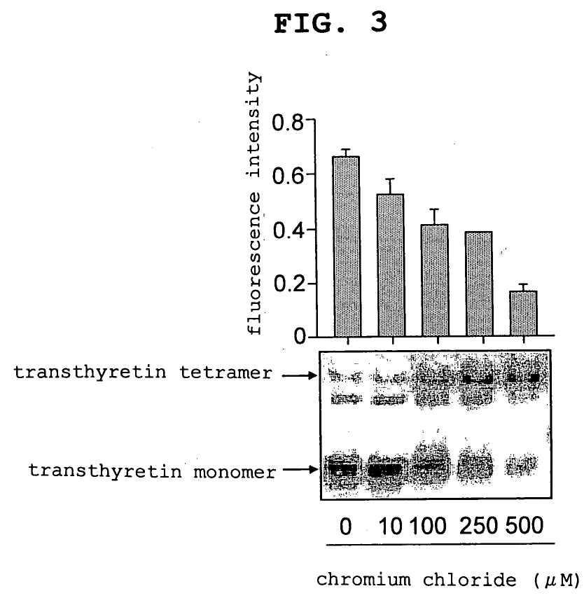

Using a differential scanning calorimeter, changes in the thermal stability during structural conversion from the transthyretin tetramer to the transthyretin monomer in the presence of the trivalent chromium ion were measured. As a measurement sample, a transthyretin-containing solution, prepared to obtain a concentration of 25 μM using 150 mM sodium chloride containing 500 μM chromium chloride and a 20 mM phosphate buffer solution, was used. The pH of the buffer solution was adjusted to a pH necessary for each experiment, using sodium hydroxide or hydrochloric acid. Measurements were conducted by injecting 1.5 ml of the sample solution to a measurement cell after degassing at room temperature for 5 minutes. The measurement conditions involved the use of a model MC-2 differential scanning calorimeter manufactured by MicroCal at a scanning rate of 1° C./min in a temperature range of 15° C. to 115° C.; during the measurement, the system was pressurized with 2 atm of nitrogen to prevent bubbling due to heat. Note that the measurement cell was washed with concentrated nitric acid for each sample. Measurement results of the amount of heat and transition temperature are shown in FIG. 2 and FIG. 3.

### EXAMPLE 3

(1) Test of Administration of Trivalent Chromium Ion to Transthyretin Transgenic Mouse

Administration of the trivalent chromium ion to a transgenic mouse incorporating a human mutant transthyretin gene having the 30th valine residue of transthyretin mutated to a methionine residue was conducted by intraperitoneally injecting 3.5 μg of a chromium chloride solution per gram of mouse weight, which is the one-sixth dosage of the LD50 value, daily. One week after administration initiation, mouse blood was recovered from the tail vein and allowed to stand at 4° C. overnight, and the serum component was isolated and used as the sample.

(2) Measurement of Stability of Transthyretin Tetramer of Transthyretin Transgenic Mouse

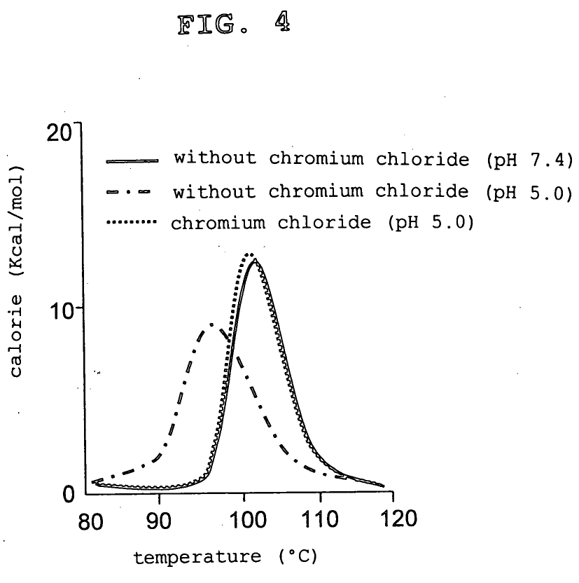

The stability of the transthyretin tetramer in a 60 μg sample recovered from the blood of the transgenic mouse was measured using Western blotting. The primary antibody used for Western blotting was an anti-human mutant transthyretin antibody (1:2000); after a primary antibody reaction, a secondary antibody reaction was conducted using a peroxidase-labeled anti-rabbit secondary antibody (1:10000). For detection of the antibody reaction, the ECL reagent was used. Results of detection of the transthyretin tetramer of the transgenic mouse are shown in FIG. 4.

### EXAMPLE 4

(1) Amyloid Fiber Production Test

A transthyretin-containing solution was dissolved in a buffer solution to obtain a concentration of 0.2 mg/mL, 0, 10 or 50 μM chromium chloride was added, and 0 or 360 nM thyroid hormone was added to each solution. The pH of the buffer solution was adjusted to a pH necessary for each experiment using sodium hydroxide or hydrochloric acid. The above-described solution was incubated at 37° C. to produce amyloid fiber.

(2) Measurement of Amount of Amyloid Fiber Produced

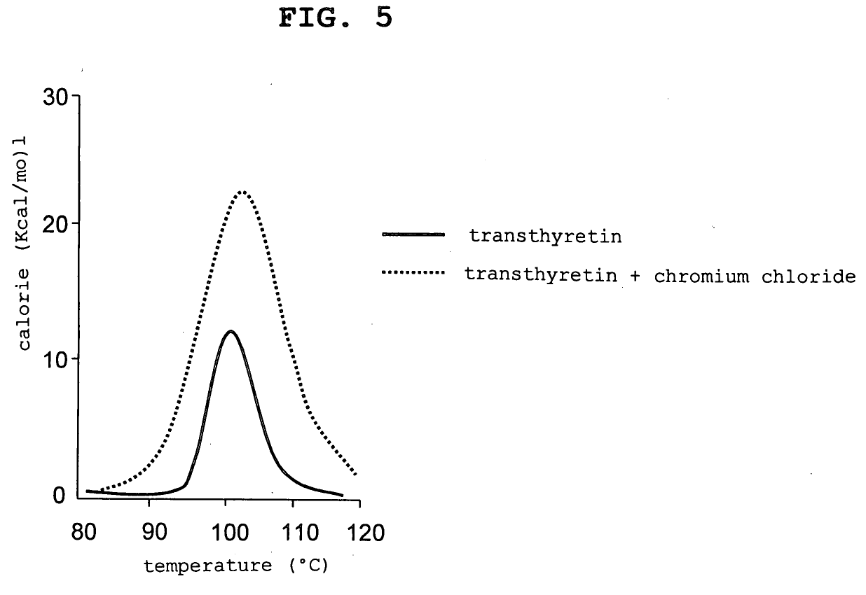

Using Thioflavine T (Wako Pure Chemical Industries, Ltd.), the amount of amyloid fiber produced was measured. As a measurement sample, the transthyretin-containing solution after incubation was diluted to obtain a transthyretin concentration of 2.5 μg/mL, supplemented with 10 μM Thioflavine T and 50 mM glycine, adjusted to pH 9.0, and used. Within 1 minute after preparation of the measurement sample, fluorescence at 482 nm excited by 450 nm excitation light was measured with the fluorescence spectrophotometer F-4500 at 25° C. using 1 ml of the sample. Note that both for the excitation side and for the fluorescence side, the measurement was conducted with a 5 nm slit width. Measurement results of fluorescence intensity are shown in FIG. 5.

### EXAMPLE 5

(1) Amyloid Fiber Production Test

A recombinant transthyretin-containing solution (10 mg/ml) was dissolved in a buffer solution to obtain a concentration of 0.2 mg/mL, 0, 10, 100 or 500 μM chromium chloride was added, and 0 or 360 nM thyroid hormone was added to each solution. The pH of the buffer solution was adjusted to a pH necessary for each experiment using sodium hydroxide or hydrochloric acid. The above-described solution was incubated at 37° C. to produce amyloid fiber.

(2) Measurement of Amount of Amyloid Fiber Produced

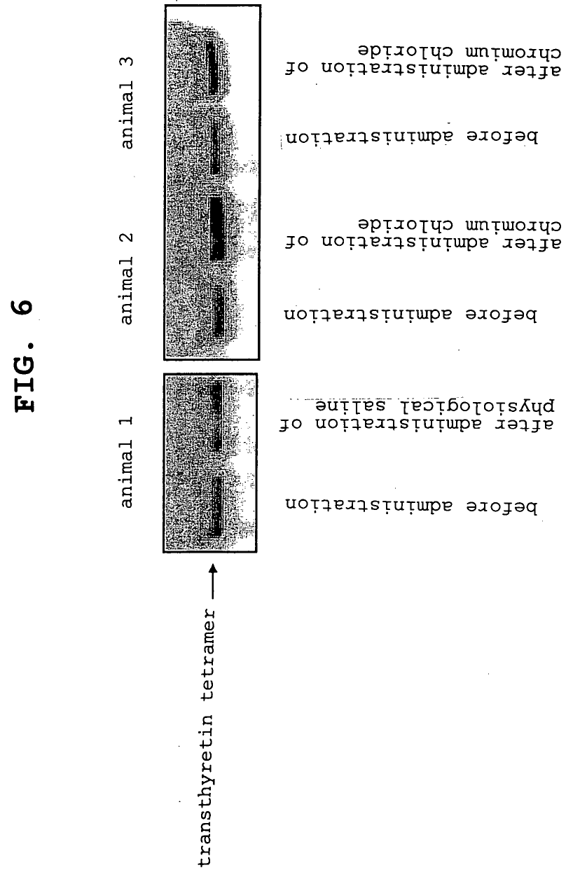

Using Thioflavine T (Wako Pure Chemical Industries, Ltd.), the amount of amyloid fiber produced was measured. As a measurement sample, the transthyretin-containing solution after incubation was diluted to obtain a transthyretin concentration of 2.5 μg/mL, supplemented with 10 μM Thioflavine T and 50 mM glycine, adjusted to pH 9.0, and used. Within 1 minute after preparation of the measurement sample, fluorescence at 482 nm excited by 450 nm excitation light was measured with the fluorescence spectrophotometer F-4500 at 25° C. using 1 ml of the sample. Note that both for the excitation side and for the fluorescence side, the measurement was conducted with a 5 nm slit width. Measurement results of fluorescence intensity are shown in FIG. 6.

### EXAMPLE 6

(1) Amyloid Fiber Production Test

A transthyretin-containing solution was dissolved in a buffer solution to obtain a concentration of 0.2 mg/mL, 0, 0.36, 1 or 10 μM diflunisal was added, and 0 or 10 μM chromium chloride was added to each solution. The pH of the buffer solution was adjusted to a pH necessary for each experiment using sodium hydroxide or hydrochloric acid. The above-described solution was incubated at 37° C. to produce amyloid fiber.

(2) Measurement of Amount of Amyloid Fiber Produced

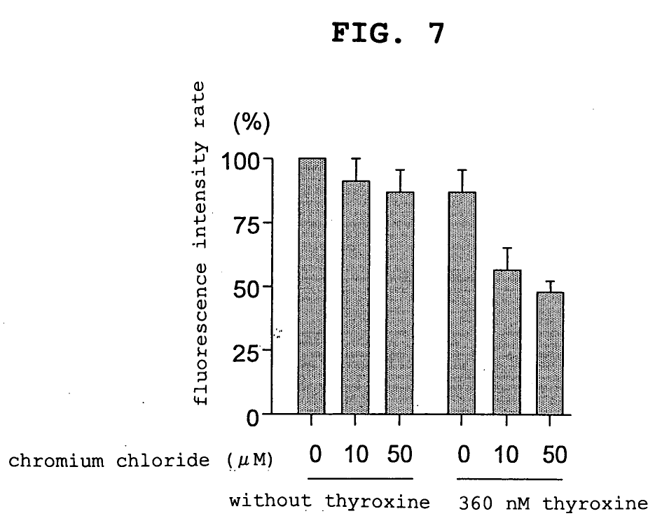

Using Thioflavine T (Wako Pure Chemical Industries, Ltd.), the amount of amyloid fiber produced was measured. As a measurement sample, the transthyretin-containing solution after incubation was diluted to obtain a transthyretin concentration of 2.5 μg/mL, supplemented with 10 μM Thioflavine T and 50 mM glycine, adjusted to pH 9.0, and used. Within 1 minute after preparation of the measurement sample, fluorescence at 482 nm excited by 450 nm excitation light was measured with the fluorescence spectrophotometer F-4500 at 25° C. using 1 ml of the sample. Note that both for the excitation side and for the fluorescence side, the measurement was conducted with a 5 nm slit width. Measurement results of fluorescence intensity are shown in FIG. 7.

### INDUSTRIAL APPLICABILITY

The pharmaceutical of the present invention is useful as a medicine for the prophylaxis and/or treatment of amyloidosis, and can particularly be used as a highly effective pharmaceutical for familial amyloid polyneuropathy (FAP), for which no therapies are available other than symptomatic therapies and liver transplantation.

1. A pharmaceutical for the prophylaxis and/or treatment of amyloidosis, which comprises a compound comprising a trivalent chromium ion as an active ingredient.

2. The pharmaceutical of claim 1, wherein amyloidosis is familial amyloid polyneuropathy.

3. The pharmaceutical of claim 1, wherein the compound comprising the trivalent chromium ion is a fatty acid chromium salt.

4. The pharmaceutical of claim 3, wherein the fatty acid is picolinic acid.

5. A pharmaceutical to suppress decomposition of a mutant transthyretin in amyloidosis, which comprises a compound comprising a trivalent chromium ion as an active ingredient.

6. A method of preventing or treating amyloidosis in a mammal, which method comprises administering a pharmaceutical composition to a mammal in which amyloidosis needs to be prevented or treated, wherein the pharmaceutical composition comprises a compound comprising a trivalent chromium ion as an active ingredient.

7. The method of claim 6, wherein the amyloidosis is familial amyloid polyneuropathy.

8. The method of claim 7, wherein the compound comprising the trivalent chromium ion is a fatty acid chromium salt.

9. The method of claim 8, wherein the fatty acid is picolinic acid.

10. The method of claim 6, wherein the compound comprising the trivalent chromium ion is a fatty acid chromium salt.

11. The method of claim 10, wherein the fatty acid is picolinic acid.

12. The method of claim 6, wherein the mammal is a human.

13. The method of claim 12, wherein the amyloidosis is familial amyloid polyneuropathy.

14. The method of claim 13, wherein the compound comprising the trivalent chromium ion is a fatty acid chromium salt.

15. The method of claim 14, wherein the fatty acid is picolinic acid.

16. The method of claim 12, wherein the compound comprising the trivalent chromium ion is a fatty acid chromium salt.

17. The method of claim 16, wherein the fatty acid is picolinic acid.

18. The method of claim 6, wherein amyloidosis is treated in a mammal.

19. A method of suppressing decomposition of a mutant transthyretin in amyloidosis in a mammal, which method comprises administering a pharmaceutical composition to a mammal in which there is decomposition of a mutant transthyretin in amyloidosis, wherein the pharmaceutical composition comprises a compound comprising a trivalent chromium ion as an active ingredient.

20. The method of claim 19, wherein the mammal is a human.
